(19) United States
(12) Patent Application Publication
(10) Pub. No.: US 2004/0175348 A1
(43) Pub. Date: Sep. 9, 2004

(54) Chitosan-containing powder

(76) Inventors: Tomoaki Kawaguchi, Miyazaki (JP); Hisako Maeda, Miyazaki (JP)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/485,187

(22) PCT Filed: Aug. 7, 2002
(86) PCT No.: PCT/JP02/08052

(30) Foreign Application Priority Data
Aug. 7, 2001 (JP) 2001-239060

(51) Int. Cl. C08B 37/08; A61K 7/06; A61K 7/11

(52) U.S. Cl. 424/70.13; 536/20

(57) ABSTRACT

A chitosan-containing powder which contains chitosan particles formed by dissolving chitosan in an edible organic acid and spray-drying the solution. The molar ratio of the amount of the chitosan to that of the edible organic acid to be used for dissolving the chitosan may be about from 1/0.9 to 1/0.8. The molecular weight of the chitosan to be dissolved in the edible organic acid may be about 10,000 to 300,000. The degree of deacetylation of the chitosan to be dissolved in the edible organic acid may be about 90% or higher. The chitosan-containing powder has a higher chitosan content than conventional ones.

### BACKGROUND OF THE INVENTION

This invention relates to chitosan-containing powder which is made from chitosan, one of natural polysaccharides, and more particularly it relates to chitosan-containing powder with a high concentration of chitosan that is easily taken and good for human health and the like.

It has been already medically proven that chitin and chitosan activate a macrophage of living organisms. It has been also proven that ingestion of chitosan is effective against many diseases. Chitin and chitosan are natural polysaccharides. Chitin is obtained from shells of crabs or prawns, and chitosan is obtained by deacetylating chitin or exists as bio-constituents of natural microorganisms.

One of the chemical characteristics of chitosan is solubility only in a diluted acid and insolubility in water and alcohol. Chitosan-solution produced by dissolving chitosan in a common organic acid, such as vinegar, lactic acid, citric acid or the like, is inappropriate to be taken as food because the solution tastes too bitter and has high viscosity. In order to dissolve chitosan, the same amount of organic acid as that of chitosan is required. Therefore, there have been only chitosan-made foods in the form of a tablet, a capsule or powder itself, powder of or in which has never been dissolved in an organic acid. However, these tablets, capsules and powder are hard to be absorbed when they are taken orally.

A Japanese patent application, JP 2-310296, discloses a sort of chitosan-made jelly food produced by dissolving chitosan in red malt. This food also tastes bitter. Moreover, during a long-term storage, this jelly food gels day by day, and it makes it more difficult to be taken as food.

Another Japanese patent application, JP 4-123391, discloses chitosan-made food produced by dissolving chitosan powder in an essence of red malt and an edible organic acid and then drying and re-powdering the solution in order to make it easier to be taken as food. This invention is provided to solve the problems, such as poor digestibility of the conventional chitosan-made foods when taken orally and the bitterness. Hence, an object of this conventional invention is to increase ingestion of chitosan by processing it to be more digestible when orally taken. However, the concentration of chitosan in obtained tablets and granules is liable to be low, so a large amount of intake of the tablets or the granules should be required to receive the effectiveness of chitosan. In addition, the chitosan content of these tablets and granules is also liable to decrease day by day as well as their color is liable to change.

Therefore, an object of the present invention is to provide chitosan-containing powder which has a higher chitosan content ratio than conventional ones.

### BRIEF SUMMARY OF THE INVENTION

To solve the above problems, the present invention has the following technical features.

1. Chitosan-containing powder according to the present invention is characterized in that it is obtained by dissolving chitosan in an edible organic acid and spray-drying the solution.

By spray-drying the solution, prepared by dissolving the chitosan in the edible organic acid, the chitosan-containing powder with a higher chitosan content ratio is obtained because the moisture and the excess organic acid in the solution are effectively evaporated therefrom.

As the edible organic acid, for example, vinegar, malic acid, vitamin C or the like can be used. A powdering process by spray-drying can be carried out in various kinds of ways. After the spray-drying, the dried powder can be processed into food substance in various forms for easy oral intake, such as a granule, a tablet, a capsule and the like.

2. The molar ratio of the amount of the chitosan to that of the edible organic acid dissolving the chitosan may be about 1/0.9 to 1/0.8.

This particular molar ratio does not decline the pH level of the chitosan-containing powder, obtained by spray-drying, when the powder is dissolved in water, so that the bitterness can be reduced.

This molar ratio also makes it possible to obtain the chitosan-containing powder having a relatively high chitosan content because the chitosan can be dissolved with the minimum amount of the organic acid required to dissolve the chitosan, so that even a small amount of oral intake of the powder is enough to activate the physiological activity of the chitosan.

3. The molecular weight of the chitosan dissolved in the edible organic acid may be about 10,000 to 300,000.

This particular molecular weight makes it possible to prepare chitosan solution having a chitosan concentration of 15% or higher. Accordingly, the bitterness can be reduced without using red malt.

4. The degree of deacetylation of the chitosan dissolved in the edible organic acid may be about 90% or higher.

This particular degree makes it possible to dissolve the chitosan in the edible organic acid at the molar ratio mentioned in the above section 2, so that the bitterness can be reduced without using red malt.

5. The chitosan content in the powder, obtained by spray-drying, may be about 30% or more.

This particular chitosan content makes it possible to receive the effectiveness of the chitosan with a small amount of oral intake.

6. The concentration of the chitosan dissolved in the edible organic acid may be about 15 w/w % or higher.

This particular chitosan concentration facilitates controlling the solid concentration of the solution, adding base materials to the solution, and spray-drying the solution when the solution is diluted with water, so that the spray-dried chitosan-containing powder can be obtained at low cost.

7. The molar ratio of the amount of the chitosan contained in the chitosan-containing powder obtained by the spray-drying to that of the edible organic acid dissolving the chitosan may be 1/0.9 or lower.

This particular molar ratio moderates the bitterness of the obtained powder and a decline of the pH level at the time of dissolving the powder. Accordingly, the powder can be used for plants and animals which are vulnerable to the pH level.

8. A method for producing chitosan-containing powder according to the present invention includes the steps of dispersing chitosan in water to make a chitosan solution, adding an edible organic acid to the solution to dissolve the chitosan by stirring the solution, filtering the solution to remove impurities therefrom, and spray-drying the solution to gain chitosan-containing powder.

In the above method, spray-drying the solution makes it possible to control the amount of base materials, such as dextrin, to be added to the solution and also makes it possible to evaporate the excess organic acid in the solution, so that the chitosan content ratio of the powder becomes higher.

9. The chitosan-containing powder according to the present invention can be added to, for example, health foods or any other food as well as animal feed or materials for farming. Even if a smaller amount of the chitosan-containing powder is added compared to that of conventional ones, the higher concentration of chitosan can be obtained.

Moreover, the chitosan-containing powder according to the present invention is suitable for use in the fields of agriculture (for growth promotion for vegetables and defense against insects), animal husbandry (for strengthening immune system of animals), fisheries industry (for strengthening immune system of fish) and so on.

Additionally, this highly concentrated chitosan-containing powder has high stability and hardly changes in quality while being stored.

### DESCRIPTION OF THE PREFERED EMBODIMENTS

Embodiments of the present invention are described below.

### EXAMPLE 1

2 kg of chitosan, made by KITOSAN FOOD INDUSTRY CO., LTD., having the average molecular weight of about 50,000 was added to 8 kg of water and kept suspended therein. 1 kg of lactic acid was further added, and the mixture was stirred for 30 minutes to dissolve. At this point, the chitosan solution has a chitosan concentration of 19% and a viscosity of 6,000 cps. In addition, the amount of the lactic acid was 0.0111 kmol while the amount of the chitosan was 0.0124 kmol; namely, the ratio of chitosan to lactic acid was 1/0.90. Subsequently, 30 kg of water was further added to dilute the solution, from which impurities had been removed by filtering, and the solution was spray-dried by a spray drier to obtain chitosan-containing powder. The spray-drying was carried out under the following conditions: the temperature of heated air was 190. degree. C.; the temperature of exhausted air was 85. degree. C.; and the fluid feed rate was 200 L/hr. As a result, 2 kg of the chitosan-containing powder having a chitosan concentration of 83% was obtained.

### EXAMPLE 2

30 kg of water in which 5 kg of dextrin had been dissolved was added to the chitosan solution prepared in Example 1, having a chitosan concentration of 19% and a viscosity of 6,000 cps. Subsequently, the solution was spray-dried by the spray drier to obtain dried chitosan-containing powder. The spray-drying was carried out under the same condition as Example 1. As a result, 7.5 kg of the chitosan-containing powder having a chitosan concentration of 43% was obtained.

### EXAMPLE 3

26 g of valine and 9.5 g of wax were added to 5 kg of the chitosan-containing powder obtained in Example 1, so as to make the powder in a form of a tablet. The chitosan content was 78%.

### EXAMPLE 4

The chitosan-containing powder obtained in Example 2 was filled into hard capsules in size 1, so as to make the powder in a form of a capsule. 380 mg of the chitosan-containing powder was filled into each capsule, and the chitosan content was 43%.

### EXAMPLE 5

The chitosan-containing powder obtained in Example 1 was filled into the hard capsules in size 1, so as to make the powder in a form of a capsule. 380 mg of the chitosan-containing powder was filled into each capsule, and the chitosan content was 80%.

### COMPARATIVE EXAMPLE 1

2 kg of lactic acid and 2 kg of red malt essence were added to 8 L of water and mixed well, and then 2 kg of chitosan, made by YAEGAKI BIO-INDUSTRY INC., was further added and stirred for 30 minutes to dissolve. The chitosan solution was filtered to remove impurities therefrom and freeze-dried. Subsequently, the freeze-dried yield was powdered. As a result, 13.3 kg of the chitosan-containing powder having a chitosan concentration of 15% was obtained.

### COMPARATIVE EXAMPLE 2

1 kg of lactose, 0.3 kg of sugar ester and 0.2 kg of tricalcium phosphate were added to the chitosan-containing powder obtained in Comparative Example 1, so as to make the powder in a form of a tablet. As a result, 11.5 kg of the yields having a chitosan concentration of 13% was obtained.

In the meantime, the molecular weight of each chitosan used in the above Examples was calculated by a liquid chromatography (HPLC). The HPLC was carried out under the following conditions.

Columns: Shodex Ohpak SB-G+SB-805HQ+SB-806HQ

Mobile phase: a buffer solution of 0.5M acetic acid/0.5M sodium acetate

Flow rate: 1.0 mL/min

Temperature: 40. degree. C.

Detector: a differential refractometer (RI)

Standard samples: Shoudex Pullulan P-5, P-10, P-20, P-50, P-100, P-200, P-400 and P-800

The molecular weights of respective standard samples were 5,900, 11,800, 22,800, 47,300, 112,000, 212,000, 404,000 and 788,000.

The molecular weight of each chitosan was calculated by using the molecular weight of glucosamine residue of 161.

Also, the concentration of each chitosan was calculated by the colloidal titration method. The concentration of each organic acid was respectively calculated by the titration method used for quantitative determination of the organic acids.

According to the above embodiments, spray-drying the solution made it possible to control the amount of the base materials, such as dextrin, to be added to the solution and also made it possible to evaporate the excess organic acid, so that the chitosan-containing powder having a higher chitosan content than conventional ones was obtained.

Since the present invention is composed as described above, and the moisture and the excess organic acid in the solution are effectively evaporated therefrom by spray-drying the solution, it is possible to provide chitosan-containing powder having a high chitosan content ratio.

We claim:

1. Chitosan-containing powder formed by spray-drying a solution of an edible organic acid wherein chitosan is dissolved.

2. The chitosan-containing powder according to claim 1, wherein a molar ratio of an amount of the chitosan to that of the edible organic acid dissolving the chitosan is about 1/0.9 to 1/0.8.

3. The chitosan-containing powder according to claim 1 or 2, wherein a molecular weight of the chitosan dissolved in the edible organic acid is about 10,000 to 300,000.

4. The chitosan-containing powder according to claim 1 or 2, wherein a degree of deacetylation of the chitosan dissolved in the edible organic acid is about 90% or higher.

5. The chitosan-containing powder according to claim 1 or 2, wherein a chitosan content of the powder obtained by the spray-drying is about 30% or more.

6. The chitosan-containing powder according to claim 1 or 2, wherein a concentration of the chitosan dissolved in the edible organic acid is about 15 w/w % or higher.

7. The chitosan-containing powder according to claim 1 or 2, wherein a molar ratio of an amount of the chitosan contained in the powder obtained by the spray-drying to that of the edible organic acid dissolving the chitosan is about 1/0.9 or less.

8. A method for producing chitosan-containing powder comprising the steps of:
dispersing chitosan in water to make a chitosan solution;
adding an edible organic acid to the solution to dissolve the chitosan by stirring the solution;
filtering the solution to remove impurities therefrom; and
spray-drying the solution to gain chitosan-containing powder.
